(19) United States
(12) Patent Application Publication
(10) Pub. No.: US 2026/0276636 A1
(43) Pub. Date: Sep. 17, 2026

(54) METHOD FOR SPECIFIC AND RAPID QUANTIFICATION OF THE TOTAL VIABLE BACTERIAL COUNT IN RAW MILK

(71) Applicant: National Institute of Metrology, Beijing (CN)

(72) Inventors: Zhiwei SUI, Beijing (CN); Meng WANG, Beijing (CN); Siyuan LIU, Beijing (CN); Ziquan WANG, Beijing (CN)

(73) Assignee: National Institute of Metrology, Beijing (CN)

(21) Appl. No.: 18/873,992

(22) PCT Filed: Mar. 27, 2024
(86) PCT No.: PCT/CN2024/083965

§ 371 Date: Dec. 11, 2024

(30) Foreign Application Priority Data
May 8, 2023 (CN) 202310510437.9

(51) Int. Cl. G01N 33/569; G01N 15/075; G01N 33/04; G01N 33/58

(52) U.S. Cl. CPC G01N 33/56911; G01N 15/075; G01N 33/04; G01N 33/56938; G01N 33/582; G01N 2333/195; G01N 2333/245; G01N 2333/31; G01N 2333/32

(57) ABSTRACT

A method for specifically, rapidly and quantitatively detecting the total viable bacterial count in raw milk, comprises adding a fluorescent probe specific for viable bacteria, a reagent regulating bacterial cell division and a treatment solution to raw milk for a labeling reaction to obtain an initial labeled sample; and detecting particle fluorescent signal in the initial labeled sample using a high-resolution flow cytometer to achieve rapid quantitative detection of the total viable bacterial count; and the total viable bacterial count detected has a good linear correlation with that detected by the traditional plate counting method. The method for rapidly detecting the total viable bacterial count in raw milk can specifically label the viable bacteria in raw milk without being interfered by other biological cells, and the growth activity of the labeled bacteria can be accurately traced to the results of the plate counting method.

This application claims the priority of Chinese Patent Application No. 202310510437.9, filed with the China National Intellectual Property Administration on May 8, 2023, and titled with “METHOD FOR SPECIFIC AND RAPID QUANTIFICATION OF THE TOTAL VIABLE BACTERIAL COUNT IN RAW MILK”, the disclosure of which is hereby incorporated by reference in its entirety.

### FIELD

The present disclosure relates to the field of food microbial detection, and in particularly to a method for specifically, rapidly and quantitatively detecting the total viable bacterial count in raw milk.

### BACKGROUND

The total viable bacterial count in raw milk is an internationally recognized hygiene indicator of raw milk production. The quality of raw milk directly affects the trading price and the quality of later milk. A lower total viable bacterial count in raw milk indicates a lower degree of external contamination during the collection process of raw milk, purer ingredients, and fewer decomposed nutrients. Unfortunately, raw milk is prone to rapid spoilage and can pose a potential threat to human health as the rich nutrients provide an ideal environment for microorganisms to reproduce quickly. At present, the total viable bacterial count in raw milk is typically detected using the plate counting method in “GB 4789.2-2010 National Food Safety Standard Food microbiological examination-Aerobic plate count”. However, this method requires 72 hours of culture to obtain the count result, which is time-consuming, cumbersome, and may not meet the practical needs of both buyers and sellers for rapid detection to perform product pricing and delivery.

Currently, common rapid detection methods for the total viable bacterial count include the ready-to-use Microbiological Test Paper, ATP detection method, and resazurin reduction method. These methods can achieve rapid detection of the total viable bacterial count based on the metabolic activity, ATP content, and respiratory activity in viable bacteria. However, the total viable bacterial count is indirectly characterized by these chromogenic methods, and can only be obtained by calculation according to an accurate standard curve. In addition, raw milk contains a large number of yeasts, molds and somatic cells, and these biological cells can also produce a chromogenic reaction. Therefore, these methods fail to achieve specific and accurate detection of the number of viable bacteria. Flow cytometry enables accurate absolute counting of all labeled cells in a sample by counting the photoelectric signals produced by individual cells. Accurate measurement of the total viable bacterial count in a milk sample can be achieved by suitable fluorescence characterization of cell activity. Currently, the most widely used methods in the industry, such as Bactoscan FC and BactoCount, use the ethidium bromide (EB) as a DNA fluorescent dye to stain cells for analysis. However, EB labels both viable and dead bacteria, as well as yeast, molds and somatic cells indiscriminately, so these methods cannot achieve a specific analysis of the number of viable bacteria. In conclusion, there is an urgent need for a method that can specifically label viable bacteria and measure their total number in raw milk.

D-alanine is an important raw material in the synthesis of bacterial peptidoglycan, which is a unique cellular structure of bacteria. Viable bacteria constantly synthesize and metabolize peptidoglycan to facilitate cell growth and response to external stimuli. Therefore, fluorescent D-amino acids (FDAA) such as D-alanine can be used to specifically label viable bacteria, allowing for the absolute quantification of the total viable bacterial count in a sample when combined with flow cytometry. At present, FDAA is mostly used for qualitative and semi-quantitative analysis of viable bacteria. Research on accurate quantitative analysis of viable bacteria through FDAA labeling is still in its early stages. In addition, the labeling process of bacteria with FDAA often takes 4-6 h, during which time the bacteria continue to grow and divide, making it challenging to convert the measured number of labeled bacteria to the initial number in the sample. In conclusion, in order to establish a rapid and accurate method for measuring the total viable bacterial count in raw milk, it is necessary to determine the feasibility of FDAA-based quantitative analysis of the number of viable bacteria. Additionally, it is important to develop inhibition techniques of bacterial cell division that can efficiently inhibit cell division during the labeling process. This will aim to achieve an accurate measurement of the initial total viable bacterial count in the sample.

### SUMMARY

In view of this, the present disclosure provides a method for specifically, rapidly and quantitatively detecting the total viable bacterial count in raw milk to solve the problem in the existing detection methods, which are time-consuming and unable to specifically determine the absolute number of viable bacteria in complex samples.

In order to achieve the above purpose, the present disclosure provides the following technical solutions.

A method for specifically, rapidly and quantitatively detecting the total viable bacterial count in raw milk is provided in this application, comprising
step 1, adding a fluorescent probe specific for viable bacteria, a reagent regulating bacterial cell division and a treatment solution to raw milk for a labeling reaction to obtain an initial labeled sample; and
step 2, detecting particle fluorescent signal in the initial labeled sample using a high-resolution flow cytometer to achieve rapid quantitative detection of the total viable bacterial count.

Wherein, the fluorescent probe specific for viable bacteria is a fluorescent probe that only labels viable bacteria, the reagent regulating bacterial cell division is an antibiotic or antibiotic cocktail that efficiently inhibits bacterial cell division without damaging bacterial viability, and the treatment solution is a protease that digests protein particles in the raw milk.

It is to be explained that in step 1, all the viable bacteria in the raw milk are labeled using a fluorescent probe specific for viable bacteria, and the cell division during the labeling of the viable bacteria is inhibited using a reagent regulating bacterial cell division, while a treatment solution is used to degrade impurities in the raw milk.

Further, the fluorescent probe specific for viable bacteria is a fluorescently labeled D-amino acid that accurately characterizes bacterial growth activity and has a structure of

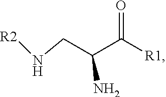

and a derivative thereof;
wherein R1 is a derivatized group and R2 is a fluorescent group.

The fluorescent probe specific for viable bacteria of the present disclosure can specifically labeling bacteria and will not label yeast or somatic cells.

As an example, the fluorescent probe specific for viable bacteria is preferably a fluorescently labeled amino D-alanine or derivatives thereof. The fluorescent group R2 is selected from the group consisting of carboxyfluorescein (FAM), tetramethylrhodamine (TAMRA), Cy5, and a combination thereof; and the amino D-alanine or derivatives thereof is selected from the group consisting of amino D-alanine, amino D-alanine amide, amino D-alanine ester, and a combination thereof.

Further, the reagent regulating bacterial cell division is selected from two or more of nalidixic acid, pipemidic acid, ciprofloxacin, levofloxacin, moxifloxacin, lomefloxacin, and nemonoxacin. The reagent regulating bacterial cell division can efficiently inhibit bacterial cell reproduction (less than 1.4-fold increase in bacterial number after treatment), while maintaining high viability (more than 94% bacterial survival rate after treatment).

Further, in step 1, the fluorescent probe specific for viable bacteria has a concentration of 100-1000 μM.

Further, in step 1, the reagent regulating bacterial cell division has a concentration of 0.1-128 μg/mL.

Further, in step 1, the labeling reaction is performed at 37° C. for 10-360 min in the dark.

Further, in step 2, the high-resolution flow cytometer has an optical resolution of 30-100 nm, a detection voltage of 300-500 V, an injection volume of 10-1000 μL, and an analysis time of 10-100 s.

Further, the high-resolution flow cytometer is used at a fluorescent wavelength of 499-540 nm.

The mathematical model between the number of fluorescently labeled particles and the total viable bacterial count in this application is a linear relationship, with a correlation coefficient R² greater than 0.98.

The present disclosure has the following advantages.
(1) The method for specifically, rapidly and quantitatively detecting the total viable bacterial count in raw milk of the present disclosure has the characteristics of simple pretreatments, short time consumption and high accuracy;
(2) The method for specifically, rapidly and quantitatively detecting the total viable bacterial count in raw milk of the present disclosure can specifically determine the number of viable bacteria in raw milk without interference from other biological cells such as yeast, mold and somatic cells;
(3) The method for specifically, rapidly and quantitatively detecting the total viable bacterial count in raw milk of the present disclosure can accurately characterize bacterial viability. The principle of using D-alanine to characterize bacterial viability is consistent with that of using traditional plate counting methods, and the obtained results has good traceability and comparability with the plate counting method results in the national standard;
(4) The method for specifically, rapidly and quantitatively detecting the total viable bacterial count in raw milk of the present disclosure can accurately characterize bacterial viability with a short measurement time. All detection can be completed within 2.5 hours without additional pretreatments, and the obtained results correspond well to those from the plate method. The efficiency of detecting the total viable bacterial count in raw milk can be significantly improved.

### BRIEF DESCRIPTION OF DRAWINGS

For more clearly illustrating embodiments of the present disclosure or technical solutions in the prior art, the drawing referred to for describing the embodiments or the prior art will be briefly described hereinafter. Apparently, the drawings in the following description are only exemplary. For those skilled in the art, other drawings may be obtained based on the provided drawings without exerting any creative efforts.

Structures, scales, dimensions, and the like as shown in the drawings are drawn in coordination with the contents disclosed in the specification, and are intended for being understood and apprehended by a skilled person rather than putting a limitation on conditions of implementing examples of the present disclosure, and therefore have no actual/real significance in techniques. Any structural modification, change in proportional relationship, or adjustment in dimensions, when not affecting an effect or a purpose of examples of the present disclosure, shall fall within the scope covered by technical content disclosed herein.

FIG. 1 shows microscope images of four species of bacteria labeled by the fluorescently labeled amino acid provided in Example 1 of the present disclosure; wherein in each image, the left shows cells under normal observation, and the right shows fluorescent labeled cells with fluorescent signals.

FIG. 2 shows the analysis results of the labeling effect of the fluorescently labeled amino acid on four species of bacteria by flow cytometry provided in Example 1 of the present disclosure.

FIG. 3 shows the results of the fluorescently labeled amino acid on viable bacteria under optimized conditions provided in Example 2 of the present disclosure.

FIG. 4 shows the proportions of viable bacteria specifically labeled by fluorescently labeled amino acid provided in Example 3 of the present application.

FIG. 5 shows the analysis results of stability of the fluorescently labeled amino acid for labeling viable bacteria provided in Example 3 of the present disclosure.

FIG. 6 shows microscope images of bacteria specifically labeled by the fluorescently labeled amino acid provided in Example 4 of the present disclosure.

FIG. 7 shows the optimized compounding results of the bacterial cell division inhibitor provided in Example 5 of the present application.

FIG. 8 shows the optimized compounding results of fluorescently labeled amino acids provided in Example 6 of the present application.

FIG. 9 shows the results of determining the detection range of the total viable bacterial count in raw milk according to the newly established method provided in Example 7 of the present application.

FIG. 10 shows the correlation analysis between the newly established method provided in Example 8 of the present disclosure and the plate counting method.

### DETAILED DESCRIPTION

The following specific examples illustrate the embodiments of the present disclosure. Those familiar with the technology can easily understand the other advantages and effects of the present disclosure from the content disclosed in this specification. It is apparent that the described examples are only a part of the examples according to the present disclosure, rather than all of the examples. All the other examples obtained by those skilled in the art based on the examples in the present disclosure without any creative work fall into the scope of the present disclosure.

The “viable bacteria” in the present disclosure refers to the viable bacteria in the raw milk.

### Example 1

In order to investigate whether fluorescently labeled D-alanine can efficiently label bacteria, carboxyfluorescein-labeled amino-D-alanine (represented by FADA) was used in this Example. After incubating FADA with bacteria of representative species in raw milk, the labeling effect of FADA on the bacterial samples was qualitatively analyzed using fluorescence microscopy, and the specific labeling efficiency was further analyzed accurately using a flow cytometer.

### I. Materials and Methods

1. Staphylococcus aureus (Deposit number: ATCC 29213), Escherichia coli (Deposit number: ATCC 10798), Lactococcus lactis (Deposit number: ATCC 19257), and Bacillus subtilis (Deposit number: ATCC 6633). The four bacterial strains are the dominant bacteria or the important indicator and pathogenic bacteria in raw milk.
2. Carboxyfluorescein-labeled amino-D-alanine (represented by FADA), purchased from Hangzhou Chinese peptide Biochemical Co., LTD.
3. Labeling method. After the four bacterial strains were activated on nutrient agar plates, a single colony was picked, inoculated to TSB medium (10 g/L peptone, 3 g/L beef extract powder, and 5 g/L sodium chloride), incubated at 37° C. for 6 h to the mid-logarithmic stage, and then centrifuged at 8000 rpm for 5 min to collect the bacterial cells. The collected bacterial cells were suspended in fresh TSB medium at a bacterial suspension concentration of approximately 10⁷ cells/mL. 1 mL of bacterial suspension was added to 2 mL of TSB medium, and FADA with a final concentration of 300 μM was added. The resulting mixture was mixed well and incubated at 37° C. for 6 h. After incubation, the bacterial cells were washed twice with PBS buffer and resuspended, and the suspension was used for later analysis.
4. Fluorescence microscopy observation method. The fluorescently labeled bacterial suspension was collected. 10 μL of bacterial suspension was added onto a slide to prepare an observation sample, which was observed using a 100× oil lens and FITC fluorescence channel. Brightfield images and fluorescence field images were recorded separately.
5. Flow cytometry. Measurement and analysis were performed using an Apogee A50 flow cytometer. The proportion of fluorescently labeled cells was measured and analyzed at an excitation wavelength of 488 nm, a flow rate of 3-25 μL/min and a detection time of 10-100 s through green fluorescence channel. The sample not incubated with FADA was used as the negative control.

### II. Experimental Results

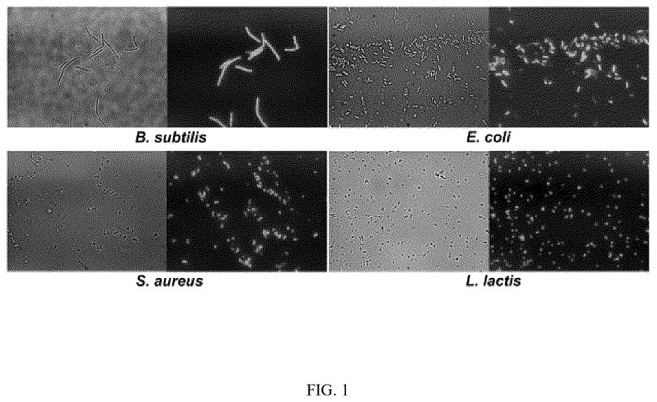

As shown in FIG. 1, after the four representative bacteria species in raw milk, B. subtilis, E. coli, S. aureus, and L. lactis, were incubated with FADA and then observed under fluorescence microscope, all cells in the field of view were found fluorescently labeled, indicating that FADA could efficiently label bacterial cells.

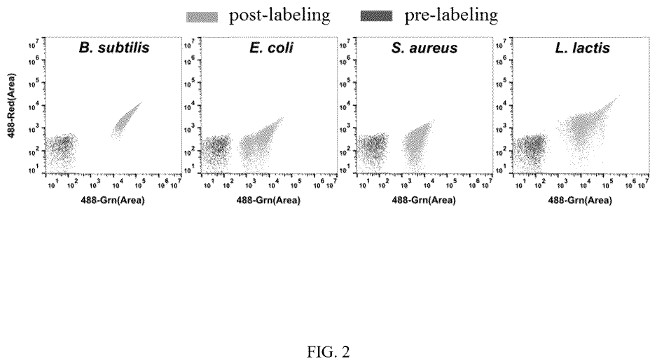

The labeling efficiency was further analyzed by flow cytometry, and the results are shown in FIG. 2. Compared with the initial samples without fluorescent labeling, the four species of bacterial cells were all fluorescently labeled and could be clearly distinguished from the unlabeled samples, with a labeling efficiency of >98.5%, indicating that the vast majority of viable bacteria were efficiently labeled, and the number of viable bacteria in the sample could be accurately counted in combination with flow cytometry.

This technical feature was used in the method for rapidly detecting the total viable bacterial count in raw milk of the present disclosure, which laid a theoretical foundation for the study of efficient labeling and accurate counting of bacteria.

### Example 2

In order to further improve the labeling efficiency of FADA for the four species of bacteria, shorten the labeling time and improve the ability to measure the total viable bacterial count in raw milk, the concentration of FADA and labeling time for bacterial samples were optimized in this example.

### I. Materials and Methods

1. Optimization of labeling conditions. After the four bacterial strains were activated on nutrient agar plates, a single colony was picked, inoculated to TSB medium (10 g/L peptone, 3 g/L beef extract powder, and 5 g/L sodium chloride), incubated at 37° C. for 6 h to the mid-logarithmic stage, and then centrifuged at 8000 rpm for 5 min to collect the bacterial cells. The collected bacterial cells were suspended in fresh TSB medium at a bacterial suspension concentration of approximately 10⁷ cells/mL. 1 mL of bacterial suspension was added to 2 mL of TSB medium, and FADA with a final concentration of 300 μM, 400 μM, and 500 μM was added. The resulting mixture was mixed well and incubated at 37° C. for 3-5 h. After incubation, the bacterial cells were washed twice with PBS buffer and resuspended, and the suspension was used for later analysis.
2. The total number of cells and the number of fluorescently labeled cells in the samples were analyzed using a flow cytometer, and the fluorescent labeling efficiency was calculated. Measurement and analysis were performed using an Apogee A50 flow cytometer. The proportion of fluorescently labeled cells was measured and analyzed at an excitation wavelength of 488 nm, a flow rate of 3-25 μL/min and a detection time of 10-100 s through green fluorescence channel.

### II. Experimental Results

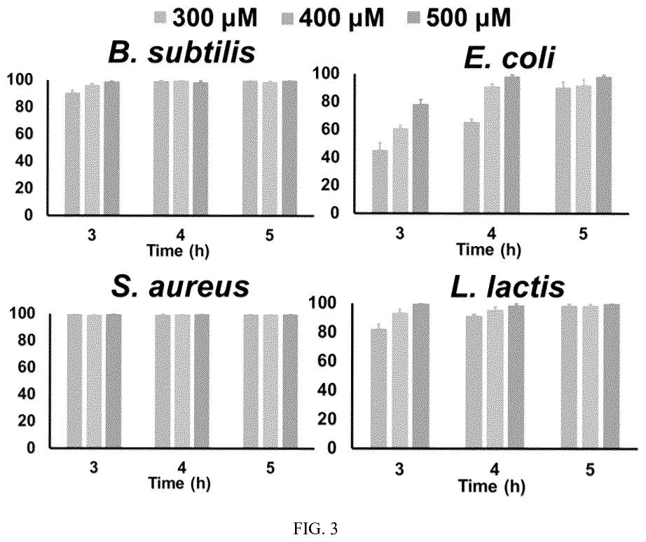

In order to improve the labeling efficiency of FADA on target bacteria, the concentration of FADA and labeling time were optimized in this example. The results are shown in FIG. 3. The labeling efficiency of FADA on the four species of bacteria was improved as the concentration of FADA increased, while the labeling efficiency of the same concentration of FADA on the same bacteria was improved as the labeling time increased. After analysis, it was found that after the four species of bacteria were incubated with 500 μM of FADA for 4 h, all bacteria were efficiently labeled with a labeling efficiency of more than 98.5%.

This example shows that FADA could efficiently label most of the bacteria under optimized labeling conditions, which could be used in combination with flow cytometry to accurately count viable bacteria.

### Example 3

In order to further verify the feasibility of combining FADA with a flow cytometer to count the number of viable bacteria in a sample, the stability of FADA for labeling viable bacteria was analyzed in this example.

### I. Materials and Methods

1. Culture and labeling of bacterial strains. After the four bacterial strains were activated on a nutrient agar plate, a single colony was picked, inoculated to TSB medium (10 g/L peptone, 3 g/L beef extract powder, and 5 g/L sodium chloride), incubated at 37° C. for 6 h to the mid-logarithmic stage, and then centrifuged at 8000 rpm for 5 min to collect the bacterial cells. The collected bacterial cells were suspended in fresh TSB medium at a bacterial suspension concentration of approximately 10⁷ cells/mL. 1 mL of bacterial suspension was added to 2 mL of TSB medium, and FADA with a final concentration of 500 μM was added. The resulting mixture was mixed well and incubated at 37° C. for 4 h. After incubation, the bacterial cells were washed twice with PBS buffer and resuspended for later use.
2. Preparation of a sample of dead bacterial cells. The suspension of viable bacteria was centrifuged, resuspended in an equal volume of 75% aqueous isopropanol, left at room temperature for 30 min, then centrifuged at 8000 g for 5 min and washed twice with an equal volume of sterile PBS buffer to prepare a suspension of dead bacteria with a concentration of approximately 10⁷ cells/mL.
3. Labeling stability test of FADA. The suspension of FADA-labeled viable bacteria and the corresponding suspension of unlabeled dead bacteria treated with isopropanol were mixed in a ratio of 1:1, and then incubated at room temperature for 2 h. The number of fluorescently labeled viable bacteria was counted using a flow cytometer. Measurement and analysis were performed using an Apogee A50 flow cytometer. The proportion of fluorescently labeled cells was measured and analyzed at an excitation wavelength of 488 nm, a flow rate of 3-25 μL/min and a detection time of 10-100 s through green fluorescence channel.
4. Fluorescence decay test of FADA label. After the suspension of FADA-labeled bacteria was left in the dark for 20 h, the changes in fluorescence intensity of the sample at 0 h and 20 h were analyzed using a flow cytometer.

### II. Experimental Results

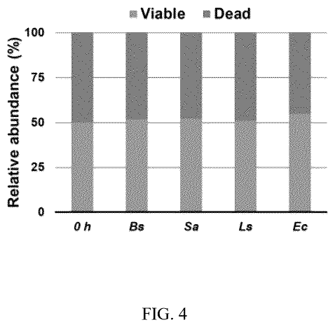

The ability of FADA to specifically label viable bacteria with a long-term stability is a key to use FADA in a rapid detection method for the total viable bacterial count in raw milk. In this example, the viable bacterial sample fluorescently pre-labeled with FADA was incubated with the dead bacterial sample without fluorescent signals in a 1:1 ratio, and the fluorescent signals were detected using a flow cytometer. The results are shown in FIG. 4. All fluorescent signals of bacteria accounted for half of the total bacteria, and the number of fluorescent signals was the same as the number of pre-labeled viable bacteria, indicating that FADA will not non-specifically migrate from viable bacteria to dead bacteria, and FADA can accurately and specifically label viable bacteria.

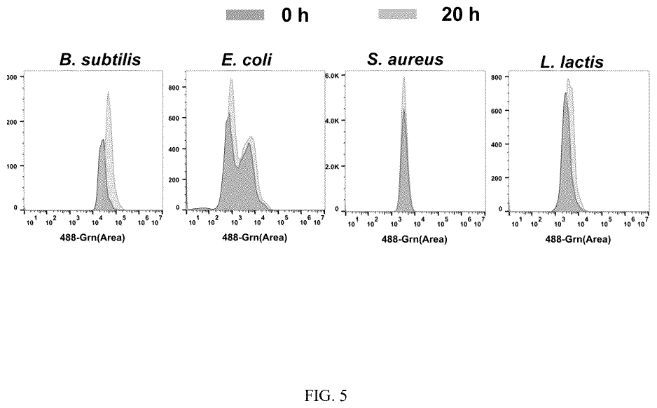

To test the labeling stability of FADA, in this example, the sample of FADA-labeled viable bacteria was left in the dark for 20 h, and the fluorescence intensity was compared to that of the sample at 0 h. The results are shown in FIG. 5. After 20 h of incubation, the signal of the FADA-labeled bacteria did not show any obvious fluorescence decay, indicating that FADA could be used in labeling up to 20 h and subsequent analysis experiments.

### Example 4

To investigate whether FADA specifically label viable bacteria in a sample and does not label yeast and somatic cells commonly found in milk, Staphylococcus aureus was used to study the specific labeling effect of FADA on bacteria in this example.

### I. Materials and Methods

1. Activation of bacterial strain. After Staphylococcus aureus was activated on a nutrient agar plate, a single colony was picked, inoculated to TSB medium (10 g/L peptone, 3 g/L beef extract powder, and 5 g/L sodium chloride), incubated at 37° C. for 6 h to the mid-logarithmic stage, and then centrifuged at 8000 rpm for 5 min to collect the bacterial cells. The collected bacterial cells were suspended in fresh TSB medium at a bacterial suspension concentration of approximately 10⁷ cells/mL.
2. Viable Saccharomyces cerevisiae (China Center of Industrial Culture Collection, CICC 1251) and Cow-derived fresh Leukocytes were separately mixed with Staphylococcus aureus, added FADA with a final concentration of 500 μM, and cultured at 37° C. for 6 h in TSB medium.
3. The fluorescence of bacteria-yeast and bacteria-leukocyte mixed samples was observed in bright field and green fluorescent field using a fluorescence microscopy.

### II. Experimental Results

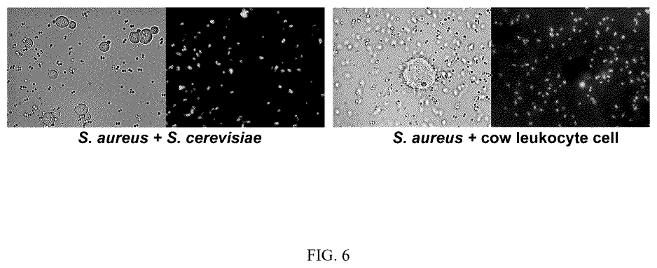

In order to verify the labeling specificity of FADA for viable bacteria, the common biological cells in raw milk were simulated, and bacteria were separately incubated with yeast and cow somatic cells for FADA labeling in this example. The results are shown in FIG. 6. In the bright field of the fluorescence microscope, S. aureus, yeast and cow leukocyte cells were obviously visible; while in the green fluorescence channel of the fluorescence microscope, only S. aureus can be observed to be fluorescently labeled, and yeast and somatic cells were not labeled. These results indicate that FADA could specifically bind to bacteria in a complex sample and achieve accurate labeling of viable bacteria.

When this technical feature was used in the rapid detection method for the total viable bacterial count in raw milk of the present disclosure, compared to other viability labeling stains, it can rapidly label viable bacteria without interference from other living biological cells in the sample to accurately detect the number of viable bacteria and achieve the accurate measurement of the total viable bacterial count in a complex sample.

### Example 5

The labeling process of viable bacteria by FADA requires 4 h, during which the bacteria constantly replicate and divide, so the number of viable bacteria measured by using a flow cytometer after labeling is the number of viable bacteria after cell division, and the initial number of viable bacteria in the sample is unknown. In order to measure the initial number of viable bacteria in the sample according to the fluorescent signal after labeling, a bacterial cell division inhibitor was developed in this example.

### I. Materials and Methods

1. Culture of bacterial strains. After the four bacterial strains were activated on a nutrient agar plate, a single colony was picked, inoculated to TSB medium (10 g/L peptone, 3 g/L beef extract powder, and 5 g/L sodium chloride), incubated at 37° C. for 6 h to the mid-logarithmic stage, and then centrifuged at 8000 rpm for 5 min to collect the bacterial cells. The collected bacterial cells were suspended in fresh TSB medium at a bacterial suspension concentration of approximately 10⁷ cells/mL.
2. Antibiotics. Quinolones antibiotics that can inhibit cell division were selected in this example, including nalidixic acid, ciprofloxacin, moxifloxacin and levofloxacin. Antibiotics were purchased from Shanghai Yuanye Biotechnology Co., Ltd. An appropriate amount of antibiotics was weighed and dissolved in DSMO to prepare a stock solution of a cell division inhibitor, which was stored in the dark at −20° C.
3. Minimum inhibitory concentration (MIC) assay. The above four bacterial suspensions were plated in a 96-well plate, to which antibiotics were added at final concentrations of 256, 128, 64, 32, 16, 8, 4, 2, 1, 0.5, and 0.25 g/mL, and then incubated at 37° C. for 48 h. Bacterial growth was analyzed using a microplate reader, and the smallest concentration of antibiotics that could inhibit the growth of bacteria was defined as the minimum inhibitory concentration.
4. Evaluation of inhibitory effect on bacterial division. The four species of bacteria were separately inoculated into TSB medium containing 0-2.5 g/mL of ciprofloxacin and 0-16 g/mL of moxifloxacin at a final concentration of 1×10⁷ cells/mL, and incubated at 37° C. for 2 h. After centrifugation at 8000 rpm, the bacterial cells were collected and resuspended in the PBS buffer, and the changes in the number of bacteria in the sample were analyzed using the forward scatter signals on the flow cytometer.
5. Detection of bacterial viability. In order to identify an antibiotic that can efficiently inhibit bacterial division while maximally maintaining bacterial viability, after treatment with ciprofloxacin and moxifloxacin, the bacterial cells were collected by centrifugation at 8000 rpm for 5 min, washed with PBS buffer and resuspended, and then the SYTO9/PI live and dead bacterial double staining solution was added to the suspension with a final concentration of 1 μM. SYTO9 labeled all bacteria in green and PI labeled dead bacteria in red. The above labeling system was left at room temperature for 30 min in the dark, washed and centrifuged with PBS twice, and the percentages of viable and dead bacteria were analyzed using a flow cytometer. Measurement and analysis were performed using an Apogee A50 flow cytometer. At an excitation wavelength of 488 nm, a flow rate of 3-25 μL/min and a detection time of 10-100 s, the total number of bacteria was analyzed in the green fluorescence channel, and the number of dead bacteria was analyzed in the red fluorescence channel.

### II. Experimental Results

The labeling process of viable bacteria by FADA requires a long time, during which the viable bacteria will continue to reproduce and divide. Therefore, when the post-labeling results are analyzed using a flow cytometer, only the number of bacteria after division can be obtained, and the initial concentration of the viable bacteria in the sample is unknown. In order to overcome these difficulties, a bacterial cell-division inhibitor that can achieve a broad-spectrum inhibition of bacterial division while maintaining bacterial viability was developed in this example.

The resistance of four common bacteria in milk to quinolone antibiotics was determined by the MIC method, and the results are shown in Table 1. Due to the large differences between the MICs of the four species of bacteria to nalidixic acid and levofloxacin, it was difficult to carry out cell division inhibition using the same concentration, and therefore ciprofloxacin and moxifloxacin were used for later study.

TABLE 1
Resistance of four common bacteria
in milk to quinolone antibiotics

| μg/mL | B. subtilis | E. coli | S. aureus | L. lactis |
|---|---|---|---|---|
| Nalidixic acid | 128 | 4 | 64 | >256 |
| Ciprofloxacin | 1 | 0.5 | 1 | 1 |
| Moxifloxacin | 8 | 1 | 4 | 2 |
| Levofloxacin | 2 | <0.25 | <0.25 | 0.5 |

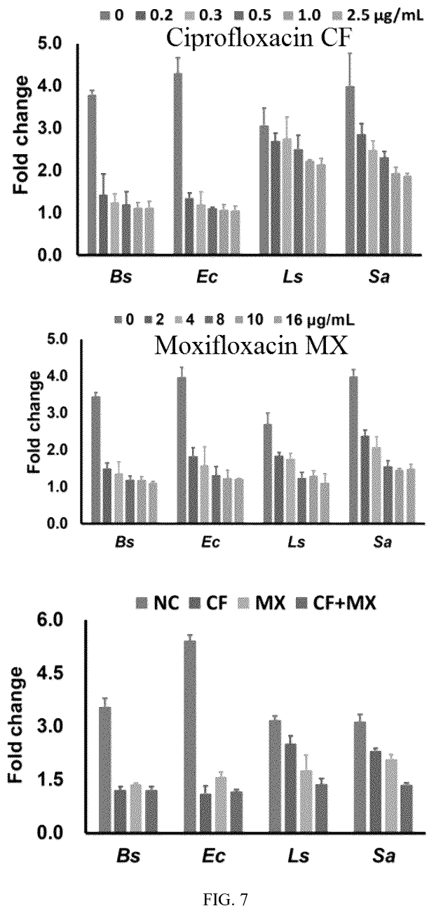

In order to determine the optimal concentration for inhibition of bacterial division, the inhibitory effects of ciprofloxacin and moxifloxacin were analyzed in this example, and the results are shown in FIG. 7. As shown in FIG. 7, compared to the blank control without antibiotics, efficient inhibition of cell division of the four species of bacteria can be achieved by 1 μg/mL of ciprofloxacin and 8 μg/mL of moxifloxacin after 2 h of treatment. The results of the bacterial viability analysis show that Bacillus subtilis was sensitive to ciprofloxacin, and the viability remained at 79.7% after antibiotic treatment; while Staphylococcus aureus was sensitive to moxifloxacin, and the viability remained at 88.1% after treatment. These results indicated that although the above antibiotics can inhibit cell division well, they seriously damaged cell viability and cannot be used in the present disclosure. In order to achieve efficient inhibition of bacterial division while maintaining cell viability, a mixture of bacterial cell division inhibitor was developed in this example: 0.5 μg/mL ciprofloxacin and 4 μg/mL moxifloxacin. After treatment with this mixture, the fold changes in cell division of all four bacteria was less than 1.4-fold, and the bacterial viability was greater than 94.6%.

The application of this technical feature in the rapid detection method for the total viable bacterial count in raw milk of the present disclosure effectively overcomes the problem that although FADA could accurately label the viable bacteria, the initial concentration of viable bacteria in the sample was difficult to know. By adding the mixture of bacterial cell division inhibitor of this example to the sample, the number of bacteria could be maintained basically unchanged during the labeling process, and after labeling, the number of fluorescently labeled bacteria could be counted by flow cytometry, to rapidly obtain the initial number of viable bacteria in the sample.

### Example 6

After using the bacterial cell division inhibitor, the cell growth and division were different from that in Example 2. Therefore, the conditions for FADA labeling were further optimized in this example to adapt the bacterial cell division inhibitor and achieve efficient labeling of viable bacteria.

### I. Materials and Methods

1. Culture of bacterial strains. After the four bacterial strains were activated on a nutrient agar plate, a single colony was picked, inoculated to TSB medium (10 g/L peptone, 3 g/L beef extract powder, and 5 g/L sodium chloride), incubated at 37° C. for 6 h to the mid-logarithmic stage, and then centrifuged at 8000 rpm for 5 min to collect the bacterial cells. The collected bacterial cells were suspended in fresh TSB medium at a bacterial suspension concentration of approximately 10⁷ cells/mL.
2. Fluorescent labeling with FADA. 1 mL of bacterial suspension was added to 2 mL of TSB medium, to which FADA at a final concentration of 500 μM and the bacterial cell division inhibitor were added, mixed well and incubated at 37° C. for 2 h. Measurement and analysis were performed using an Apogee A50 flow cytometer. The proportion of fluorescently labeled cells was measured and analyzed at an excitation wavelength of 488 nm, a flow rate of 3-25 μL/min and a detection time of 10-100 s through green fluorescence channel.
3. Fluorescent labeling with FADAA (Amino FADA). 1 mL of bacterial suspension was added to 2 mL of TSB medium, to which FADAA at a final concentration of 500 μM and the mixture of bacterial cell division inhibitor were added, mixed well and incubated at 37° C. for 2 h. Measurement and analysis were performed using an Apogee A50 flow cytometer. The proportion of fluorescently labeled cells was measured and analyzed at an excitation wavelength of 488 nm, a flow rate of 3-25 μL/min and a detection time of 10-100 s through green fluorescence channel.

### II. Experimental Results

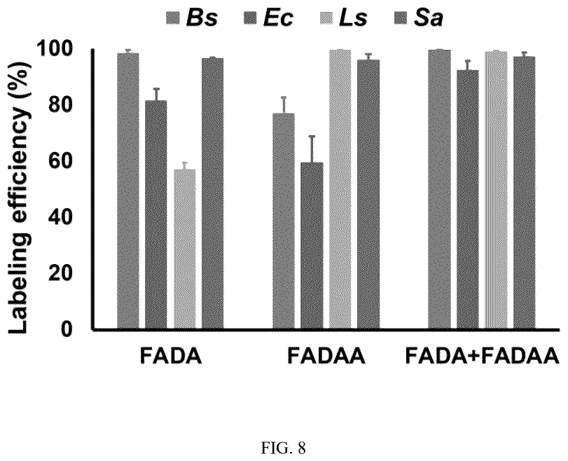

Example 5 shows that the bacterial cell division inhibitor could efficiently inhibit bacterial division within 2 h. After the bacterial cell division inhibitor was added to the culture system, although the bacteria no longer divided, they were still metabolically active, and the bacteria will continue to grow or elongate at this time, and FADA could still be used as a substrate for peptidoglycan synthesis to achieve the labeling of viable bacteria. In this case, the bacterial division was inhibited, the demand for nutrients of bacteria was reduced, and likewise the demand for FADA and the labeling time were reduced. Therefore, the labeling effect of FADA on four species of bacteria after 2 h of labeling was investigated in this example. As shown in FIG. 8, the results show that the labeling effect of FADA was less effective on E. coli (81.6%) and L. lactis (57.1%), and it was difficult to meet the requirements for accurate measurement.

In order to improve the labeling efficiency, another fluorescent marker FADAA, a D-alanine derivative studied previously, was used in this example. The results (FIG. 8) showed that FADAA had a good labeling effect on Lactococcus lactis (>96.1%), and a slightly poor labeling effect on Escherichia coli. In order to investigate whether mixing FADA and FADAA could synergistically improve the labeling effect on bacteria, a fluorescent labeling mixture was prepared by mixing FADA and FADAA at half the original concentration of each, and its labeling efficiency was analyzed using a flow cytometer. The results (FIG. 8) show that the four species of bacteria could be efficiently labeled by the fluorescent labeling mixture within 2 h under the condition of inhibitory bacterial division, and the labeling efficiencies were all >92.4%.

The application of this technical feature in the rapid detection method of the total viable bacterial count in raw milk in the present disclosure achieves rapid and accurate labeling of viable bacteria. That is, a mixture of bacterial cell division inhibitors was added to the sample to inhibit cell division during the labeling process, and a fluorescent labeling mixture was added to achieve broad-spectrum and highly efficient labeling of viable bacteria. After fluorescent labeling, the number of viable bacteria in the sample is accurately measured using a flow cytometer.

### Example 9

In order to examine the ability of the rapid test method of the present disclosure to measure the total viable bacterial count in raw milk, the effective detection range of the method was analyzed in this example.

### I. Materials and Methods

1. Culture of bacterial strains. After the four bacterial strains were activated on a nutrient agar plate, a single colony was picked, inoculated to TSB medium (10 g/L peptone, 3 g/L beef extract powder, and 5 g/L sodium chloride), incubated at 37° C. for 6 h to the mid-logarithmic stage, and then centrifuged at 8000 rpm for 5 min to collect the bacterial cells. The collected bacterial cells were suspended in fresh TSB medium at a bacterial suspension concentration of approximately 10⁹ cells/mL.
2. Preparation of artificially contaminated samples. Commercially available milk (Mengniu Dairy) was used as a food matrix to investigate and analyze the detection range of the method of the present disclosure. Compared to raw milk, commercially available milk has essentially the same main components, but with almost no viable bacteria and a relatively simple bacterial background, making it an ideal matrix for the preparation of artificially contaminated samples. The above bacterial cells were separately collected, resuspended and diluted in milk to prepare artificially-contaminated milk samples containing bacterial cells at 10³, 10⁴, 10¹, 10⁶, 10⁷, 10⁸, and 10⁹ cells/mL.
3. Fluorescent labeling of viable bacteria. 1 mL of milk sample was added to 2 mL of TSB medium, to which FADA and FADAA at final concentrations of 250 μM each, the mixture of bacterial cell division inhibitors and a protease mixture were added, mixed well and incubated at 37° C. for 2 h. Measurement and analysis were performed using an Apogee A50 flow cytometer. The proportion of fluorescently labeled cells was measured and analyzed at an excitation wavelength of 488 nm, a flow rate of 3-25 μL/min and a detection time of 10-100 s through green fluorescence channel.
4. Plate counting method. The milk samples were diluted in a 10-fold gradient, and the diluted samples (100 μL) were evenly spread on a nutrient agar plate and incubated at 37° C. for 24 h. After incubation, the number of colonies on the plate was counted and multiplied by the dilution times to obtain the original number of viable bacteria in the sample.

### II. Experimental Results

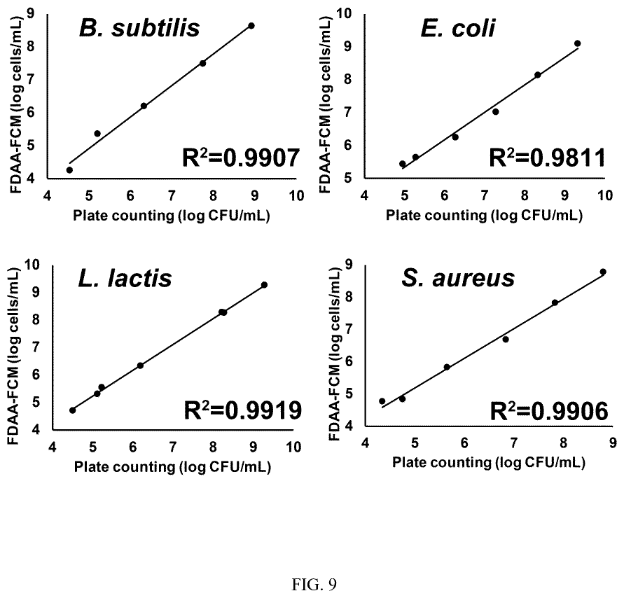

During the measurement of artificially contaminated samples, a protease mixture was used to digest protein particles during incubation and reduce the background signal, a fluorescently labeled alanine mixture was used to specifically label viable bacteria, a mixture of bacterial cell division inhibitors was used to maintain the number of viable bacteria essentially unchanged, and a flow cytometer was used to accurately count the fluorescently labeled viable bacteria. Overall, the measurement of the number of viable bacteria in a sample could be completed within 2.5 h in this example. As shown in FIG. 9, the results show that the method of the present disclosure had a good linear correlation with the plate counting method in the detection range of 10⁴-10⁹ cells/mL, with a correlation coefficient R² of >0.98. This detection limit can be in good agreement with the regulatory thresholds for dairy products in China, Europe and the United States, where the requirement for the total viable bacterial count in raw milk is less than 2×10⁶ cells/mL in China, 1×10⁵ cells/mL in the European Union, and 5×10⁵ cells/mL in the United States. Therefore, this feature in the rapid detection method of the total viable bacterial count in raw milk of the present disclosure can be practically used in the measurement of the total viable bacterial count in raw milk.

### Example 8

In order to examine the practical application effect of the method established in this application, in this example, the total viable bacterial count in real raw milk was detected by this method and the correlation between the detection results obtained by this method and those obtained by conventional plate counting method was analyzed.

### I. Materials and Methods

1. Detection of the total number of fluorescent labeled viable bacteria. 1 mL of raw milk sample was added to 2 mL of TSB medium, to which FADA and FADAA at final concentrations of 250 μM each, a mixture of bacterial cell division inhibitors and a protease mixture were added, mixed well and incubated at 37° C. for 2 h. Measurement and analysis were performed using an Apogee A50 flow cytometer. The proportion of fluorescently labeled cells was measured and analyzed at an excitation wavelength of 488 nm, a flow rate of 3-25 μL/min and a detection time of 10-100 s through green fluorescence channel.
2. Plate counting method. The raw milk samples were diluted in a 10-fold gradient, and the diluted samples (100 μL) were evenly spread on a nutrient agar plate and incubated at 37° C. for 72 h. After incubation, the number of colonies on the plate was counted and multiplied by the dilution times to obtain the original number of viable bacteria in the sample.

### II. Experimental Results

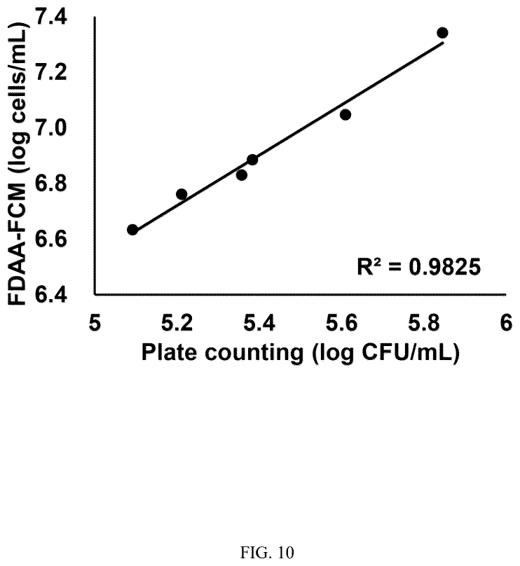

In order to investigate whether the method for rapid and accurate detecting the total viable bacterial count in raw milk of the present disclosure can detect the total viable bacterial count in real raw milk, six different batches of raw milk samples were collected from a local farm in Beijing in this example. Measurements for the raw milk samples were performed separately by the method of the present disclosure and by the traditional plate counting method. As shown in FIG. 10, the results show that the total viable bacterial count measured by the method of the present disclosure has a good linear correlation with that measured by the plate count method, with a correlation coefficient of R² of 0.9825, indicating that the method of the present disclosure can be used in the measurement for real samples. In addition, compared to the traditional plate counting method, the method of the present disclosure can obtain the total viable bacterial count within 2.5 h, which can effectively shorten the measurement time, facilitate product delivery, and contribute to the rapid microbiological evaluation of the raw milk and early warning. Compared to the existing flow cytometry, the method of the present disclosure can specifically detect the number of viable bacteria, obtains the more accurate results, does not require any additional pre-treatment operations (only the addition of protease mixture during the labeling process is required for incubation), and has a simpler operation and higher efficiency.

Cow milk has been studied in the present disclosure as an example, but raw milk of other mammals such as goat milk, donkey milk, and the like, is also within the scope of protection of the present disclosure.

Although the present disclosure has been described in detail above with general descriptions and specific examples, some modifications or improvements can be made on the basis of the present disclosure, which is apparent to those skilled in the art. Therefore, all those modifications and improvements made without departing from the spirit of the present disclosure fall within the scope of the present disclosure.

What is claimed is:

1. A method for specifically, rapidly and quantitatively detecting a total viable bacterial count in raw milk, comprising
step 1, adding a fluorescent probe specific for viable bacteria, a reagent regulating bacterial cell division and a treatment solution to raw milk for a labeling reaction to obtain an initial labeled sample; and
step 2, detecting particle fluorescent signal in the initial labeled sample using a high-resolution flow cytometer to achieve rapid quantitative detection of the total viable bacterial count;
wherein the fluorescent probe specific for viable bacteria is a fluorescent probe that only labels viable bacteria, the reagent regulating bacterial cell division is an antibiotic that efficiently inhibits bacterial cell division without damaging bacterial viability, and the treatment solution is a protease that digests protein particles in the raw milk.

2. The method according to claim 1, wherein the fluorescent probe specific for viable bacteria is a fluorescently labeled D-amino acid that accurately characterizes bacterial growth activity and has a structure of 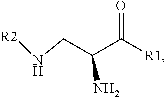
and a derivative thereof,
wherein R1 is a derivatized group and R2 is a fluorescent group.

3. The method according to claim 1, wherein the reagent regulating bacterial cell division is selected from two or more of nalidixic acid, pipemidic acid, ciprofloxacin, levofloxacin, moxifloxacin, lomefloxacin, and nemonoxacin.

4. The method according to claim 1, wherein in step 1, the fluorescent probe specific for viable bacteria has a concentration of 100-1000 μM.

5. The method according to claim 1, wherein in step 1, the reagent regulating bacterial cell division has a concentration of 0.1-128 μg/mL.

6. The method according to claim 1, wherein in step 1, the labeling reaction is performed at 37° C. for 10-360 min in the dark.

7. The method according to claim 1, wherein in step 2, the high-resolution flow cytometer has an optical resolution of 30-100 nm, a detection voltage of 300-500 V, an injection volume of 10-1000 μL, and an analysis time of 10-100 s.

8. The method according to claim 7, wherein the high-resolution flow cytometer is used at a fluorescence detecting wavelength of 499-540 nm.
